(19) United States
(12) Patent Application Publication
(10) Pub. No.: US 2007/0020650 A1
(43) Pub. Date: Jan. 25, 2007

(54) Methods for detecting proteins

(76) Inventor: Avak Kahvejian, Boston, MA (US)

Correspondence Address: EDWARDS & ANGELL, LLP, P.O. BOX 55874, BOSTON, MA, 02205, US

(21) Appl. No.: 11/396,945

(22) Filed: Apr. 3, 2006

Related U.S. Application Data
(60) Provisional application No. 60/667,790, filed on Apr. 1, 2005.

(51) Int. Cl. C12Q 1/68; G01N 33/53

(52) U.S. Cl. 435/6; 435/7.1

(57) ABSTRACT

The invention provides methods for detecting antigens comprising forming an antibody/antigen complex in which the antibody is coupled to a polynucleotide having a known sequence. The sequence of the polynucleotide is identified in order to identify the antibody, thereby detecting the antigen.

### REFERENCE TO RELATED APPLICATIONS

This application claims the benefit of and priority to U.S. provisional application No. 60/677,790, filed Apr. 1, 2005, the entirety of which is hereby incorporated by reference.

### TECHNICAL FIELD OF THE INVENTION

The invention generally relates to methods for detecting antigens on a support, and more particularly, to methods for identifying a protein using an antibody coupled to a polynucleotide of a known sequence.

### BACKGROUND OF THE INVENTION

Antibodies are produced by B lymphocytes through an immune reaction as a result of antigenic stimulation. An antibody is capable of specifically reacting with an antigen, such as a protein, to achieve aggregation, sedimentation, or neutralization of toxicity. The portion of the antigen to which an antibody binds is referred to as an epitope. Generally, a single type of antigen has multiple epitopes. Antibodies have the property of specifically and strongly binding with antigens, so that the antibodies are widely used for detection of antigens.

Techniques are being developed that enable simultaneous measurement of multiple molecules using solid supports, flat chips, or membranes as carriers on which biopolymers such as nucleic acids, antibodies, or antigens, are immobilized. Many important biomarkers of cancers, infectious diseases, or biochemical reactions have very low concentrations in blood, body fluids or tissues, so that they are difficult to detect by conventional immunoassays. Especially for those samples with little and limited amounts of an antigen or antigens at extremely low concentrations, higher sensitivity and specificity are required.

Therefore, a need remains for improved methods of detecting, identifying and enumerating proteins.

### SUMMARY OF THE INVENTION

The invention provides methods for detecting antigens, such as proteins, by exposing an antigen to a capture agent, such as an antibody, that is coupled to a polynucleotide of a known sequence. The capture agent specifically binds to the antigen, thereby producing a support bound capture agent/antigen complex. The support bound capture agent/antigen complex contains the polynucleotide of known sequence attached to the capture agent. The complex is detected by sequencing the polynucleotide attached to the capture agent. The identity of the antigen is determined based upon the sequence of the polynucleotide attached to the capture agent. Methods of the invention may be conducted in solution or, preferably, are conducted using a support-bound antigen or antibody as described below.

Methods of the invention are useful for detecting a multiplicity of the same or different antigens and may further comprise the step of enumerating antigens on the surface. In order to distinguish different antigens, different capture agents, each specific for a different antigen, are used. Each capture agent is coupled to a different polynucleotide of known sequence. Therefore, sequencing each polynucleotide present in the resulting agent/antigen complexes allows the unique identification of the capture agent to which the polynucleotide is attached.

Single molecule sequencing techniques are particularly useful for determining the sequence of the polynucleotide tag. For example, nucleic acid tags are attached to a specific antibody and the complex is then placed on a surface such that at least some of the complexes are individually optically resolvable. Sequencing comprises exposing the capture agent/antigen complexes to a nucleic acid primer that is complementary to a portion of the polynucleotide portion of the polynucleotide-conjugated capture agent. The polynucleotide serves as the template, and labeled nucleotides are added sequentially to the primer in a template-dependent manner. The nucleotides may be labeled with, for example, a fluorescent label and may be detected individually upon incorporation into the primer. Methods of the invention may further comprise removing the label from the nucleotide upon detection of the label. Only as many nucleotides as are required to detect the polynucleotide, or to differentiate one polynucleotide sequence from another (where more than one polynucleotide sequence is present), need be sequenced. For example, methods of the invention may comprise determining only one nucleotide to detect the polynucleotide, or may comprise determining the sequence for only a portion of the polynucleotide.

Any sequencing method is useful for practice of the invention. In addition to the one described above, sequencing may be conducted using optical labels and fluorescence resonance energy transfer (FRET), essentially as described in Braslavaky, et al., PNAS, 100: 3960-64 (2003), incorporated by reference herein. For example, a FRET donor molecule can be placed on the polymerase, the template, or the nucleotide to be incorporated and the FRET acceptor can be placed on any of the foregoing on which the donor is not placed. Sequencing may also be accomplished in real time using a pyrosequencing, essentially as disclosed in Nordstrom, et al., Analytical Biochemistry, 282: 186-193 (2000), incorporated by reference herein. Alternatively, a “movie mode” sequencing process involves template-dependent sequencing by synthesis in which each of the four Watson-Crick bases to be added has attached thereto a different colored fluorescent label. Other sequencing methods known in the art are also contemplated as discussed below.

### BRIEF DESCRIPTION OF THE DRAWINGS

FIG. 1. is a schematic showing two different antibodies attached to two different polynucleotides of known sequence.

FIG. 2. is a schematic showing the target antigens (hexagons) of antibodies A and B, respectively, bound to a surface, and the binding of the polynucleotide-conjugated antibodies to their respective surface-bound antigen.

FIG. 3. is a schematic showing the detection of the bound antibodies and thereby detection of the surface-bound antigen by sequencing the polynucleotide portions of the polynucleotide-antibody conjugates.

### DESCRIPTION OF THE INVENTION

The invention provides methods for identifying, detecting, and quantitating antigens. Methods of the invention comprise the use of sequencing, especially at the single molecule level, in order to identify antigens. Thus, in one embodiment, the invention comprises attaching a polynucleotide to an antibody and then exposing the antibody to a substrate-bound antigen. Preferably, methods of the invention utilize single molecule nucleic acid sequencing in which attached polynucleotides are sequenced in a template-dependent manner on the surface such that each polynucleotide (and the individual nucleotides incorporated therein) is individually optically resolvable. Methods of the invention allow for a highly parallel detection and enumeration of proteins in a sample. The high-throughput nature of methods of the invention allows massively parallel processing and when used with single molecule sequencing, and allows precise identification and quantitation of proteins in a sample.

Antigens in a sample can be enumerated using a number of parallel methods. For example, in order to distinguish different antigens, a different capture agent, one specific for each antigen to be detected can be used. Each capture agent is coupled to a different polynucleotide sequence (also referred to herein as a DNA or polynucleotide tag). The nucleotide sequence of the polynucleotide is subsequently determined using sequenced by synthesis techniques, thereby identifying the polynucleotide tags, and consequently of the associated antibody and antigen. This allows detection and/or enumeration of all or most proteins in a given biological sample, and provides a digital expression profile of a cell at the protein level.

In another embodiment, the level of one or more antigens in a sample can be identified. The antigens are attached to a support such that polynucleotide-conjugated antibodies that subsequently bind the antigen are individually optically resolvable. The polynucleotide-conjugated antibody is allowed to bind the immobilized antigen and the nucleotide sequence of the polynucleotide is subsequently determined using sequenced by synthesis techniques, thereby identifying each polynucleotide-conjugated antibody that has bound to an antigen on the surface, and consequently enumerating the antigens attached to the surface. Where more than one antigen is to be detected and enumerated, different capture agents, each one specific for a different antigen can be used as described above.

Methods of the invention are amenable to various alternatives. For example, RNA can be used instead of DNA. Also contemplated are nucleic acid analogs, such as peptide nucleic acids and locked nucleic acids, among others. Nucleic acids for use in the invention may be modified at the convenience of the user in order to facilitate incorporation and subsequent detection. For example, nucleotides having a 3′ blocking group are useful for incorporation into the primer during the sequencing steps in order to control the rate of sequencing (see, e.g., U.S. Ser. No. 11/046,448, filed Jan. 28, 2005, incorporated by reference herein). Also, linker groups can be incorporated into nucleotides in order to facilitate incorporation and detection. In one embodiment, the 3′ terminus of the polynucleotide portion of the polynucleotide-conjugated antibody is blocked, thereby preventing addition of nucleotides of labeled nucleotides to the polynucleotide during the sequencing steps.

Methods and compositions of the invention are well-suited for use in single molecule sequencing techniques. The capture agent/antigen complexes formed on the surface as described above, are exposed to a primer under conditions suitable to hybridize the primer to the polynucleotide portion of the capture agent, thereby forming a template/primer duplex, where the polynucleotide portion of the capture agent is the template. A polymerase and at least one labeled nucleotide corresponding to a first nucleotide species is added. The duplexes are washed of unincorporated labeled nucleotides, and the incorporation of labeled nucleotide is detected. The polymerization reaction is serially repeated in the presence of a labeled nucleotide that corresponds to each of the other nucleotide species in order to compile a sequence of incorporated nucleotides that is representative of the complement to the template nucleic acid. Where a single polynucleotide is to be sequenced, the nucleotides can be added in order corresponding to the known sequence of the polynucleotide. Where more than one polynucleotide is to be sequenced, the nucleotides can be added in an order chosen at the convenience of the user.

The polymerization reaction is repeated as many times as necessary to complete sequencing of a desired length of the polynucleotide. Once the desired number of cycles is complete, the result is a stack of images represented in a computer database. For each spot on the surface that contained an initial capture agent/antigen duplex, there will be a series of light and dark image coordinates, corresponding to whether a base was incorporated in any given cycle. For example, if the polynucleotide sequence was TACGTACG and nucleotides were presented in the order CAGU(T), then the duplex would be “dark” (i.e., no detectable signal) for the first cycle (presentation of C), but would show signal in the second cycle (presentation of A, which is complementary to the first T in the template sequence). The same duplex would produce signal upon presentation of the G, as that nucleotide is complementary to the next available base in the template, C. Upon the next cycle (presentation of U), the duplex would be dark, as the next base in the template is G. Upon presentation of numerous cycles, the sequence of the polynucleotide would be built up through the image stack. The resulting sequence corresponds to the complement of the polynucleotide portion of the polynucleotide-conjugated antibody, which in turn corresponds to and identifies the antigen to which the polynucleotide-conjugated antibody is bound. Techniques for single molecule nucleic acid sequencing are disclosed in Braslavaky, et al., PNAS 100, 3960-3964 (2004), U.S. Ser. No. 10/852,482, filed May 24, 2004, and U.S. Patent Application No. US-2006/0019276 A1 by Harris, et al., the teachings of which are incorporated herein by reference in their entireties.

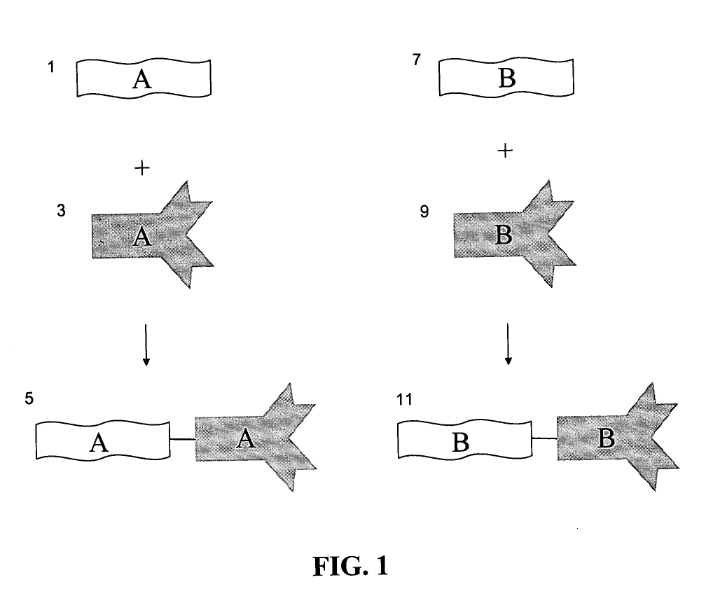

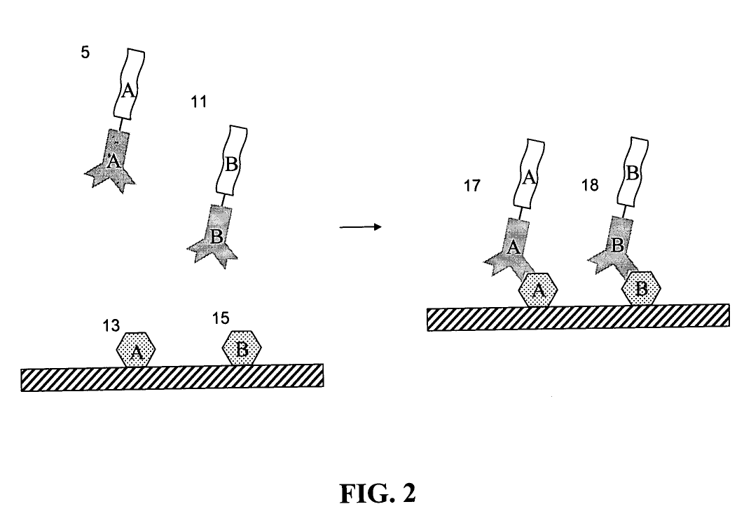

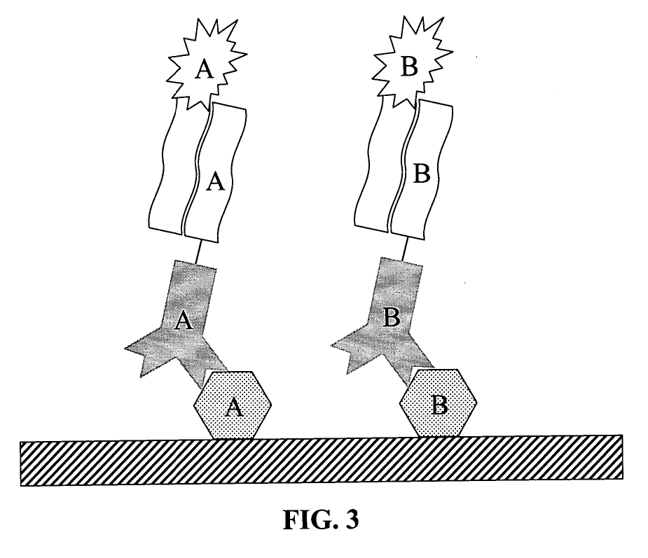

A schematic representation of the invention is shown in FIGS. 1-3. As shown in FIG. 1, a polynucleotide 1 having a known sequence (A) is attached to an antibody 3 that is capable of binding a particular antigen, forming a polynucleotide-conjugated antibody 5. Similarly, a second polynucleotide 7 having a known sequence (B) is attached to a second antibody 9 that is capable of binding a second particular antigen, forming a second polynucleotide-conjugated antibody 11. As shown in FIG. 2, the polynucleotide conjugated antibodies 5 and 11 are allowed to bind to their respective surface bound antigens 13 and 15 to form antibody/antigen complexes 17 and 18. As shown in FIG. 3, the polynucleotide portions of the antigen bound, polynucleotide-conjugated antibodies are sequenced, thereby identifying the polynucleotide portion of the polynucleotide-conjugated antibodies and their respective antibodies and antigens.

General Considerations

A. Antibodies

Antibodies for use in the present invention can be generated by methods well known in the art (see, for example, Antibodies, a Laboratory Model, E. Harlow and D. Lane, Cold Spring Harbor Laboratory Press, Cold Spring Harbor, N.Y., 1988). In addition, wide variety of antibodies are available commercially. Antigens suitable for the present invention include any molecule capable of eliciting a specific antibody that is capable of binding to the antigen. Suitable antigens include, for example, proteins, polypeptides, peptides, carbohydrates, nucleic acids, and combinations thereof.

B. Attachment of Polynucleotide Tags

Polynucleotides suitable for the present invention can be of any suitable length. In some embodiments, the polynucleotide tag can be about 10 to about 200 nucleotides in length. In other embodiments, the polynucleotide tag is about 20 in length. In still other embodiments, the polynucleotide tag is about 50 nucleotides in length. The polynucleotide tags have nucleotide sequences chosen at the convenience of the user. The polynucleotide tags can be synthesized using a number of different techniques. For example, polynucleotides having desired sequences can be synthesized chemically. In addition, polynucleotides can be synthesized using the polymerase chain reaction with suitable primers and template nucleic acid. In another embodiment, the polynucleotide can be part of a larger sequence, such as a plasmid, that is replicated in a host cell. The plasmid can be isolated from the host cell and the polynucleotide can be isolated from the plasmid using methods well know in the art. For example, the plasmid can be designed to have restriction sites flanking the polynucleotide of interest. In addition, polynucleotides having desired sequence can be obtained commercially.

Methods for coupling (also referred to herein as conjugating) the polynucleotide to the antibody antibody are known in the art. For example, as described in U.S. Pat. No. 5,219,996 to Bodmer, et al., the teachings of which are incorporated herein by reference, recombinant antibodies can be produced in which a cysteine residue has been introduced to provide a thiol group which is available for covalent binding to a desired molecule such as a polynucleotide that has been modified to include a disulfide. In another embodiment, as described in U.S. Pat. No. 5,196,066 to Bieniarz, et al. (the teachings of which are incorporated herein by reference), antibodies may be derivatized by selectively introducing sulfhydryl groups in the Fc region of the antibody, such that the antibody combining site is unaffected. In another embodiment, a polynucleotide can be conjugated to an antibody as described in U.S. Pat. No. 5,428,132 to Hirsch and Hirsch, the teachings of which are incorporated herein in their entirety.

C. Antigens

Antigens suitable for the present invention include any molecule capable of eliciting a specific antibody that is capable of binding to the antigen. Suitable antigens include, for example, proteins, polypeptides, peptides, carbohydrates, and nucleic acids. Antigens for use in the present invention can be obtained from any cellular material from an animal, plant, bacterium, fungus, or any other cellular organism. In one embodiment, antigens are obtained from viral material. Antigens can be obtained directly from an organism or from a biological sample obtained from an organism, e.g., from blood, urine, cerebrospinal fluid, seminal fluid, saliva, sputum, stool and tissue. Any tissue or body fluid specimen may be used as a source of antigens. Antigens can also be obtained from cultured cells, such as a primary cell culture or a cell line. In one embodiment, the cells from which antigens are obtained can be infected with a virus or other intracellular pathogen in order to obtain the antigens from the virus or other intracellular pathogen. Cells can be obtained, for example from biopsy material, as described, for example in U.S. Pat. No. 6,969,614 to Liotta, et al., the teachings of which are incorporated herein by reference.

Generally, antigens, such as proteins, polypeptides or peptides, can be extracted from a biological sample by a variety of techniques such as those described by U.S. Pat. No. 6,969,614, the teachings of which are incorporated herein by reference. A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is nondenaturing, can act to solubilize the sample. Detergents may be ionic or nonionic. Examples of nonionic detergents include triton, such as the Triton® X series (Triton® X-100 t-Oct-C₆H₄—(OCH₂—CH₂)ₓOH, x=9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant. Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid. In addition, proteins can be extracted from biological samples using commerically avaiable kits. Protein extracton kits for bacterial, yeast, and mammalian cells are available commercially, for example from Calbiochem (EMD Biosciences, Inc., San Diego, Calif.).

In one embodiment, antigens such as proteins or polypeptides can be treated to produce fragments for use in the present invention. Proteins and polypeptides can be fragmented, for example, by sonication, enzymatic digestion, or chemical digestion. Exemplary protocols for the aforementioned methods are well known in the art and many are detailed at Protocol Online (on the world wide web at protocol-online.org)

D. Attachment of Antigens Sample to a Surface

There are numerous methods known in the art for attaching antigens to a surface. In a one embodiment, the surface comprises an epoxide coating. Use of epoxide coated surfaces (such as glass surfaces) to immobilize proteins are described in U.S. Patent Application No. 2006/0019276 by Harris, et al., and in U.S. Pat. No. 4,071,409 to Messing, et al., the teachings of which are incorporated herein by reference. For example, proteins can be immobilized onto epoxy silane-derivatized or isothiocyanate-coated glass slides. Succinylated proteins may also be coupled to aminophenyl- or aminopropyl-derivatised glass slides, and disulfide-modified amino acids can be immobilized onto a mercaptosilanised glass support by a thiol/disulfide exchange reaction. The concentration of the antigen in the sample can be adjusted so that antigen is attached to the surface in a manner that allows polynucleotide-conjugated antibody to bind in an individually optically resolvable manner.

E. Nucleotides

Nucleotides useful in the invention include any nucleotide or nucleotide analog, whether naturally-occurring or synthetic. For example, preferred nucleotides include phosphate esters of deoxyadenosine, deoxycytidine, deoxyguanosine, deoxythymidine, adenosine, cytidine, guanosine, and uridine. Other nucleotides useful in the invention comprise an adenine, cytosine, guanine, thymine base, a xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, locked nucleic acids and any other structural moiety that can act substantially like a nucleotide or base, for example, by exhibiting base-complementarity with one or more bases that occur in DNA or RNA and/or being capable of base-complementary incorporation, and includes chain-terminating analogs. A nucleotide corresponds to a specific nucleotide species if they share base-complementarity with respect to at least one base.

Nucleotides for nucleic acid sequencing according to the invention preferably comprise a detectable label that is directly or indirectly detectable. Preferred labels include optically-detectable labels, such as fluorescent labels. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are cyanine-3 and cyanine-5. Labels other than fluorescent labels are contemplated by the invention, including other optically-detectable labels.

F. Nucleic Acid Polymerases

Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Komberg and Baker, W. H. Freeman, New York, N.Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9°Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DP1I/DP2 DNA polymerase II (Cann et al., 1998, Proc Natl Acad. Sci. USA 95:14250-->5).

While mesophilic polymerases are contemplated by the invention, preferred polymerases are thermophilic. Thermophilic DNA polymerases include, but are not limited to, ThermoSequenase®, 9°Nm™, Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof.

Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit Rev Biochem. 3:289-347(1975)).

G. Surfaces

In a preferred embodiment, antigens are attached to a substrate (also referred to herein as a surface). Polynucleotide-conjugated antibody is allowed to bind the surface-attached antigens, and the polynucleotide portion of the bound antibody is subjected to analysis by single molecule sequencing. In a preferred embodiment, the antigens are attached to the surface such that subsequently bound polynucleotide-conjugated antibodies are individually optically resolvable. Substrates for use in the invention can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring an antigen.

In one embodiment, a substrate is coated to allow optimum optical processing and antigen attachment. Substrates for use in the invention can also be treated to reduce background. Exemplary coatings include epoxides, and derivatized epoxides (e.g., with a binding molecule, such as streptavidin, isocyanate, or isothiocyatate). The surface can also be treated to improve the positioning of attached antigens for analysis. The carboxyl groups of the polyacrylic acid layer are negatively charged and thus repel negatively charged labeled nucleotides, improving the positioning of the label for detection. Coatings or films applied to the substrate should be able to withstand subsequent treatment steps (e.g., photoexposure, boiling, baking, soaking in warm detergent-containing liquids, and the like) without substantial degradation or disassociation from the substrate.

Examples of substrate coatings include, vapor phase coatings of 3-aminopropyltrimethoxysilane, as applied to glass slide products, for example, from Molecular Dynamics, Sunnyvale, Calif. In addition, generally, hydrophobic substrate coatings and films aid in the uniform distribution of hydrophilic molecules on the substrate surfaces. Importantly, in those embodiments of the invention that employ substrate coatings or films, the coatings or films that are substantially non-interfering with primer extension and detection steps are preferred. Additionally, it is preferable that any coatings or films applied to the substrates either increase antigen binding to the substrate or, at least, do not substantially impair antigen binding.

H. Detection

Any detection method may be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al., Proc. Natl. Aca. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached template nucleic acids.

A number of approaches can be used to detect incorporation of fluorescently-labeled nucleotides into the primer. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for two-dimensional imaging. TIRF microscopy uses totally internally reflected excitation light and is well known in the art. See, e.g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached template/primer complex in the presence of a polymerase. Total internal reflectance fluorescence microscopy is then used to visualize the attached polynucleotide/primer duplex and/or the incorporated nucleotides with single molecule resolution.

Certain embodiments of the invention are described in the following examples, which are not meant to be limiting.

### EXAMPLE 1

Preferred methods of the invention comprise determining the sequence of antibody-linked nucleic acid by a sequencing-by-synthesis method. Incorporated nucleotides are detected by virtue of their optical emissions after sample washing. Primers are hybridized to the polynucleotide portion of the polynucleotide-conjugated antibody. Sequencing reactions are conducted in a stepwise fashion. Reactions are conducted using Klenow fragment Exo-minus polymerase (New England Biolabs) at 10 nM (100 units/ml) and a labeled nucleotide triphosphate in EcoPol reaction buffer (New England Biolabs). Sequencing reactions takes place in a stepwise fashion. First, 0.2 μM dUTP-Cy5 and polymerase are introduced, incubated for 6 to 15 minutes, and washed out. Images of the surface are then analyzed for primer-incorporated U-Cy5. Typically, eight exposures of 0.5 seconds each are taken in each field of view in order to compensate for possible intermittency (e.g., blinking) in fluorophore emission. Software is employed to analyze the locations and intensities of fluorescence objects in the intensified charge-coupled device pictures. Fluorescent images acquired in the WinView32 interface (Roper Scientific, Princeton, N.J.) are analyzed using ImagePro Plus software (Media Cybernetics, Silver Springs, Md.). Essentially, the software is programmed to perform spot-finding in a predefined image field using user-defined size and intensity filters. The program then assigns grid coordinates to each identified spot, and normalizes the intensity of spot fluorescence with respect to background across multiple image frames. From those data, specific incorporated nucleotides are identified. Generally, the type of image analysis software employed to analyze fluorescent images is immaterial as long as it is capable of being programmed to discriminate a desired signal over background. The programming of commercial software packages for specific image analysis tasks is known to those of ordinary skill in the art. If U-Cy5 is not incorporated, the substrate is washed, and the process is repeated with dGTP-Cy5, dATP-Cy5, and dCTP-Cy5 until incorporation is observed. The label attached to any incorporated nucleotide is neutralized, and the process is repeated. To reduce bleaching of the fluorescence dyes, an oxygen scavenging system can be used during all green illumination periods, with the exception of the bleaching of the primer tag.

In order to determine a template sequence, the above protocol is performed sequentially in the presence of a single species of labeled dATP, dGTP, dCTP or dUTP. By so doing, a first sequence is compiled that is based upon the sequential incorporation of the nucleotides into the extended primer. The first compiled sequence is representative of the complement of the bound polynucleotide. As such, the sequence of the polynucleotide is easily determined by compiling a second sequence that is complementary to the first sequence.

### EXAMPLE 2

Epoxide-coated are slides were prepared for oligo attachment. Epoxide-functionalized 40 mm diameter #1.5 glass cover slips (slides) are obtained from Erie Scientific (Salem, N.H.). The slides are preconditioned by soaking in 3×SSC for 15 minutes at 37° C. Next, an aliquot of a sample that contains the antigen of interest is incubated with each slide for 30 minutes at room temperature in a volume of 80 ml. The resulting slides have antigen attached by direct amine linkage to the epoxide. The slides are then treated with phosphate (1M) for 4 hours at room temperature in order to passivate the surface. Slides are then stored in polymerase rinse buffer (20 mM Tris, 100 mM NaCl, 0.001% Triton X-100, pH 8.0) until use.

Polynucleotide-conjugated antibody is incubated with the slide under conditions suitable to allow the antibody to bind the antigen. Conditions suitable for antibody/antigen binding are described in Antibodies a Laboratory Manual by E. Harlow and D. Lane, Cold Spring Harbor Press, 1988). Unbound polynucleotide-conjugated antibody can be removed by rinsing the slide with buffer.

To sequence the polynucleotide portion of the polynucleotide-conjugated antibody, the slides are placed in a modified FCS2 flow cell (Bioptechs, Butler, Pa.) using a 50 um thick gasket. The flow cell is placed on a movable stage that is part of a high-efficiency fluorescence imaging system built around a Nikon TE-2000 inverted microscope equipped with a total internal reflection (TIR) objective. The slide is then rinsed with HEPES buffer with 100 mM NaCl and equilibrated to a temperature of 50° C. An aliquot of Cy3-labeled primer capable of hybridizing to the polynucleotide is placed in the flow cell and incubated on the slide for 15 minutes. After incubation, the flow cell is rinsed with 1×SSC/HEPES/0.1% SDS followed by HEPES/NaCl. A passive vacuum apparatus is used to pull fluid across the flow cell. The resulting slide contains polynucleotide primer duplex, the polynucleotide being conjugated to antibody that is bound to the surface-attached antigen. The temperature of the flow cell is then reduced to 37° C. for sequencing and the objective is brought into contact with the flow cell.

For sequencing, cytosine triphosphate, guanidine triphosphate, adenine triphosphate, and uracil triphosphate, each having a cyanine-5 label (at the 7-deaza position for ATP and GTP and at the C5 position for CTP and UTP (PerkinElmer)) are stored separately in buffer containing 20 mM Tris-HCl, pH 8.8, 10 mM MgSO₄, 10 mM (NH₄)₂SO₄, 10 mM HCl, and 0.1% Triton X-100, and 100 U Klenow exo⁻ polymerase (NEN). Sequencing proceeds as follows.

First, initial imaging is used to determine the positions of duplex on the epoxide surface. The Cy3 label attached to the primer is imaged by excitation using a laser tuned to 532 nm radiation (Verdi V-2 Laser, Coherent, Inc., Santa Clara, Calif.) in order to establish duplex position. For each slide only single fluorescent molecules are imaged in this step are counted. Imaging of incorporated nucleotides as described below is accomplished by excitation of a cyanine-5 dye using a 635 nm radiation laser (Coherent). 5 uM Cy5CTP is placed into the flow cell and exposed to the slide for 2 minutes. After incubation, the slide is rinsed in 1×SSC/15 mM HEPES/0.1% SDS/pH 7.0 (“SSC/HEPES/SDS”) (15 times in 60 ul volumes each, followed by 150 mM HEPES/150 mM NaCl/pH 7.0 (“HEPES/NaCl”) (10 times at 60 ul volumes). An oxygen scavenger containing 30% acetonitrile and scavenger buffer (134 ul HEPES/NaCl, 24 ul 100 mM Trolox in MES, pH6.1, 10 ul DABCO in MES, pH6.1, 8 ul 2M glucose, 20 ul NaI (50 mM stock in water), and 4 ul glucose oxidase) is next added. The slide is then imaged (500 frames) for 0.2 seconds using an Inova301K laser (Coherent) at 647 nm, followed by green imaging with a Verdi V-2 laser (Coherent) at 532 nm for 2 seconds to confirm duplex position. The positions having detectable fluorescence are recorded. After imaging, the flow cell is rinsed 5 times each with SSC/HEPES/SDS (60 ul) and HEPES/NaCl (60 ul). Next, the cyanine-5 label are cleaved off incorporated CTP by introduction into the flow cell of 50 mM TCEP for 5 minutes, after which the flow cell is rinsed 5 times each with SSC/HEPES/SDS (60 ul) and HEPES/NaCl (60 ul). The remaining nucleotide is capped with 50 mM iodoacetamide for 5 minutes followed by rinsing 5 times each with SSC/HEPES/SDS (60 ul) and HEPES/NaCl (60 ul). The scavenger is applied again in the manner described above, and the slide is again imaged to determine the effectiveness of the cleave/cap steps and to identify non-incorporated fluorescent objects. The procedure described above is then conducted 100 nM Cy5dATP, followed by 100 nM Cy5dGTP, and finally 500 nM Cy5dUTP. The procedure (expose to nucleotide, polymerase, rinse, scavenger, image, rinse, cleave, rinse, cap, rinse, scavenger, final image) is repeated exactly as described for ATP, GTP, and UTP except that Cy5dUTP is incubated for 5 minutes instead of 2 minutes. Uridine is used instead of Thymidine due to the fact that the Cy5 label is incorporated at the position normally occupied by the methyl group in Thymidine triphosphate, thus turning the dTTP into dUTP.

Once the desired number of cycles are completed, the image stack data (i.e., the single molecule sequences obtained from the various surface-bound duplex) is analyzed to determine the sequence of the polynucleotide portion of the polynucleotide-conjugated antibody.

Also according to methods of the invention, nucleic acids can be attached to the antigen and the antibodies can be bound to the surface. The invention also contemplates other alternatives that do not deviate from the scope and spirit of the invention as expressed herein.

I claim:

1. A method for detecting a protein, the method comprising the steps of:
coupling an antibody to a polynucleotide having a known sequence;
exposing said antibody to a surface-bound antigen in order to form an antibody/antigen complex on said surface;
sequencing said polynucleotide; and
identifying said antigen based upon the sequence of said polynucleotide.

2. The method of claim 1, wherein a plurality of the same antigen is bound to the surface.

3. The method of claim 1, wherein a plurality of different antigens are bound to the surface.

4. The method of claim 1, further comprising the step of enumerating said antigens on said surface.

5. The method of claim 1, wherein said polynucleotide is individually optically resolvable.

6. The method of claim 1, wherein said sequencing step comprises:
exposing said polynucleotide to a nucleic acid primer that is complementary to a portion of the polynucleotide under conditions suitable to form a duplex;
contacting said duplex with a polymerase and labeled nucleotides under conditions suitable to add said labeled nucleotide to said primer in a template-dependent manner.

7. The method of claim 6, wherein said label is a fluorescent label.

8. The method of claim 7, wherein said fluorescent label is detected individually upon incorporation into said primer.

9. The method of claim 8, wherein said label is removed from said nucleotide upon detection of said label.

10. The method of claim 8, further comprising the step of compiling a sequence of nucleotides incorporated into said primer.

11. A method for identifying an antigen, the method comprising the steps of:
(a) exposing a support-bound antigen to a known antibody coupled to a polynucleotide of a known sequence, to form an antibody/antigen complex on said support;
(b) detecting said antibody/antigen complex by detecting said polynucleotide; and
(c) identifying said antigen based upon said known antibody.

12. The method of claim 11 wherein said detecting said polynucleotide comprises performing a sequencing reaction.

13. The method of claim 12 wherein at least one nucleotide of said polynucleotide is determined.

14. The method of claim 12 wherein a sequence for at least a portion of said polynucleotide is determined.

15. The method of claim 12 wherein said performing a sequencing reaction comprises:
exposing said polynucleotide to a nucleic acid primer that is complementary to a portion of the polynucleotide under conditions suitable to form a duplex;
contacting said duplex with a polymerase and labeled nucleotides under conditions suitable to add said labeled nucleotide to said primer in a template-dependent manner.
